(19) United States
(12) Patent Application Publication
(10) Pub. No.: US 2010/0198079 A1
(43) Pub. Date: Aug. 5, 2010

(54) METHOD AND DEVICE FOR EASY ACCESS TO VASCULAR GRAFT CANNULATION SITES

(76) Inventor: John R. Ross, Bamberg, SC (US)

Correspondence Address: PASSE' INTELLECTUAL PROPERTY, LLC, 1717 BRASSFIELD RD., RALEIGH, NC, 27614, US

(21) Appl. No.: 12/758,988

(22) Filed: Apr. 13, 2010

Related U.S. Application Data
(62) Division of application No. 12/047,445, filed on Mar. 13, 2008.

(51) Int. Cl. A61B 6/00

(52) U.S. Cl. 600/476

(57) ABSTRACT

The present invention relates to a polymeric venous implant wherein at least a portion of the graft is made from a UV fluorescing polymer which allows the user to see the graft during access use. This allows the graft to be injected much easier by the ability of the technician to find the graft or a stick site on the graft.

This application is a divisional application of U.S. application Ser. No. 12/047,445 filed on Mar. 13, 2008 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to improved venous implants. In particular the present invention relates to a polymeric venous implant wherein at least a portion of the graft is made from a UV fluorescing polymer which allows the user to see the graft during access use.

2. Description of Related Art

Hemodialysis is a life-sustaining treatment for patients who have renal failure. Hemodialysis is a process whereby the patient's blood is filtered and toxins are removed using an extracorporeal dialysis machine. For hemodialysis to be effective, large volumes of blood must be removed rapidly from the patient's body, passed through the dialysis machine, and returned to the patient. A number of operations have been developed to provide access to the circulation system of a patient such that patients may be connected to the dialysis machine.

For example, the most commonly performed hemodialysis access operation is a subcutaneous placement of an arteriovenous graft, which is made from a biocompatible tube. The biocompatible tube can be made of, for instance, a fluoropolymer such as polytetrafluoroethylene. One end of the tube is connected to an artery while the other end is connected to a vein. The arteriovenous graft is typically placed either in the leg or arm of a patient.

Blood flows from the artery, through the graft and into the vein. To connect the patient to a dialysis machine, two large hypodermic needles are inserted through the skin and into the graft. Blood is removed from the patient through one needle, circulated through the dialysis machine, and returned to the patient through the second needle. Typically, patients undergo hemodialysis approximately four hours a day, three days a week.

These types of grafts typically have a number of problems in their construction and as such a number of different approaches to graft design have been developed. In U.S. Pat. No. 7,144,381 issued Dec. 5, 2006 to Gertner there is a hemodialysis system and method described with adjustable members. In U.S. Pat. No. 7,147,617 to Henderson et al. and issued Dec. 12, 2006 there is another example of an arterio-venous shunt graft. In U.S. patent application 2006/0229548 published Oct. 12, 2006 there is an arteriovenous graft system with access valve systems along with methods of using them.

While the use of these grafts substantially improves the hemodialysis process it is clear that venous graft have asset of problems associated with their use. Injection into the graft is typically accomplished by the nursing staff or worse lower level medical technicians without the ability to read ultra sound or other techniques for finding the graft to access with a needle. Accordingly, it is typical that these technicians and staff thus use either touch or a previously done “diagram” to place the needle. Because of the location and the like of the graft these personnel are almost attempting to access the graft blindly. Stick site errors result in the patient presenting complications such as pseudoaneursms, aneurysms, thrombus, clots and blockage with the possibility of total occlusion of the graft needing replacement. This is not to mention the potential pain and discomfort the patient experiences.

The resulting complications cost tens of millions of dollars in invasive treatments to cure these problems. That doesn't include lost work time and the problems associated with further surgical intervention. The vast majority of graft recipients are on public medical assistance and, as such, these problems are not only a huge financial burden on the user but also a huge cost to the US taxpayer. While there have been AV type grafts for decades now, as well as frequent improvements in the design of AV grafts, there have been no improvements in the ability to see the portion of the graft that is hidden, especially the portion underneath the skin.

Accordingly, it would be useful if there were additional methods, grafts or the like that would aid the healthcare worker in accessing vascular grafts for needle insertion.

### BRIEF SUMMARY OF THE INVENTION

In the present invention, it has been discovered that a vascular graft can be made easier to use by one trying to insert a needle in such graft. This can be accomplished by inclusion within the graft of a composition which absorbs UV light and fluoresces, i.e. a UV fluorescing composition upon exposure to a source of UV light, such as a black light.

By exposing the area where the graft is implanted to a black light the healthcare worker attempting to access the graft will be able to see the exposed portion of the graft, for example an access point or even see the graft fluorescing under the skin for finding an access point.

Accordingly, in one embodiment of the invention, there is provided an arteriovenous graft comprising a UV fluorescing material, wherein the UV fluorescing material is positioned such that upon exposure to a UV light source the graft or a portion of the graft fluoresces sufficiently to improve the visibility of the graft by a health care worker attempting to access the graft.

In yet another embodiment of the present invention there is disclosed a method for accessing an arteriovenous graft in a patient comprising:
a) selecting an arteriovenous graft comprising a UV fluorescing material wherein the UV fluorescing material is positioned such that upon exposure to a UV light source the graft or a portion of the graft fluoresces sufficiently to improve the graft visibility;
b) positioning the graft in the patient;
c) applying a UV light source to the area where the graft is positioned; and
d) identifying an access point in the graft by observing the fluorescing graft.

In yet another embodiment of the invention there is disclosed a method of accessing an arteriovenous graft in a patient comprising:
a) selecting a patient having an arteriovenous graft comprising a UV fluorescing material wherein the UV fluorescing material is positioned such that upon exposure to UV light source the graft or portion of the graft fluoresces sufficiently to improve graft visibility;
b) applying a UV light source to the area where the graft is positioned;
c) identifying a place to insert an access needle by observing the fluorescing graft.

### BRIEF DESCRIPTION OF THE DRAWINGS

FIG. 1 is a perspective view of an embodiment of the present invention where there are two bands of UV fluorescing compound.

FIG. 2 is a perspective view of an embodiment of the present invention where there is a graft which is entirely made with a UV fluorescing polymer.

FIG. 3 is a perspective view showing multiple stick sites.

FIG. 4 is a perspective view of a graft showing different UV polymers indicating front wall and back wall of the graft.

### DETAILED DESCRIPTION OF THE INVENTION

The present invention relates to the inclusion of at least a portion of an AV graft including a material which fluoresces upon application of UV light. The present invention can include just the injection sites or the entire device, which can be totally or partially under the skin during use. The present invention overcomes the limitations and problems of the prior art for those medical technicians attempting to insert a needle into an AV graft. Even though the problem has been around since the introduction of AV grafts and still exists, and technology moves at a rapid pace, no previous solution accomplishes the results of the present invention.

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

The terms “a” or “an”, as used herein, are defined as one as or more than one. The term “plurality”, as used herein, is defined as two, or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “and an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C”. An exception to this definition will occur only when a combination of elements, functions, steps or acts are in some way inherently mutually exclusive.

As used herein an arteriovenous graft is a biocompatible tube which is subcutaneously placed for access by a healthcare worker during hemodialysis. One end of the tube is connected to an artery while the other end is connected to a vein. Typically the insertion of the AV graft is by placement in the leg or arm of a patient. The biocompatible tube can be made of, for instance, a fluoropolymer such as polytetrafluoroethylene.

Blood flows from the artery, through the graft and into the vein. To connect the patient to a dialysis machine, two large hypodermic needles are inserted through the skin and into the graft. Blood is removed from the patient through one needle, circulated through the dialysis machine, and returned to the patient through the second needle. This process is often performed for over four hours a day, three times a week. It is clear that insertion of the needle through the skin and into the graft should be as accurate as possible each time because of the problems associated with poor needle insertion as described above.

As used herein, the term biocompatible UV fluorescing material relates to a material which can be incorporated in, coated on or used to make an AV graft of the present invention. These compositions are photo-chromic substances, which are known to be essentially colorless but have the property of photo-reacting to longwave ultraviolet (UV) light (about 250 to 400 nm) and can change to a variety of colors and shades. The intensity of the resulting visible color (reds, violets, blues, etc) is directly proportional to the intensity of the UV light source, i.e. the more intense the UV light the more intense the resulting visible light will be.

One method of producing the present invention is to incorporate a photochromic compound directly into the polymer matrix. The polymer can be injection molded or the like directly into the graft shape from there. Examples of plastic which could incorporate the compound for photochromic behavior include polyol(allyl carbonate)-monomers, polyacrylated, polyethylenes, polypropylenes, polyvinyl chloride, polymethylmethacrylates, cellulose acetate, cellulose triacetate, cellulose acetate propionate, cellulose acetate butyrate, polyacetal resins, acetyl cellulose, poly vinyl acetate, poly vinyl alcohols, poly urethanes, poly carbonates, polystyrenes, including copolymers and other biocompatible polymer molecules. The color of the fluorescing material will depend on the photochromic composition selected for inclusion in the present invention.

Another means of preparing the present invention is to incorporate the photochromic compound in one polymer and bind the polymer to the polymer of the graft tubing. That way a particular area could be caused to glow and not just the entire tubing or graft itself. While the photochromic compound could be included in just a portion of the tubing fabrication of separate polymer containing photochromic polymers (using the same or different polymers) would be easier to make. So, in one embodiment, only the area that a needle is to be inserted will glow. In another embodiment each of the two insertion sites would be caused to glow a different color.

As used herein, the phrase, “UV light source” would refer to a longwave UV light, in one embodiment hand held, of sufficient light intensity to cause the UV polymer to glow considering its position subcutaneously, and still be able to visually observe the UV glow of the photochromic compound beneath the skin. Such UV handheld devices are well known within the art and are mentioned for convenience.

In the use of the present invention, a patient in need of an AV graft would have a graft of the present invention surgically implanted and positioned in an appropriate place between an artery and a vein for access by a healthcare worker or technician. Once the graft of the present invention is positioned in place in a patient, the healthcare worker would turn on a high intensity UV light and shine it in the general area (an arm or leg for example) where the graft was placed and look for the appropriate glow. The worker could then, while observing the glow, insert the appropriate dialysis needles into the graft for use in dialysis of the patient. In one embodiment each site for needle insertion is color coded a different glowing color so that placement of each needle (one close to the artery and one close to the vein) can easily be identified by use of separate colors of glowing polymer for each position.

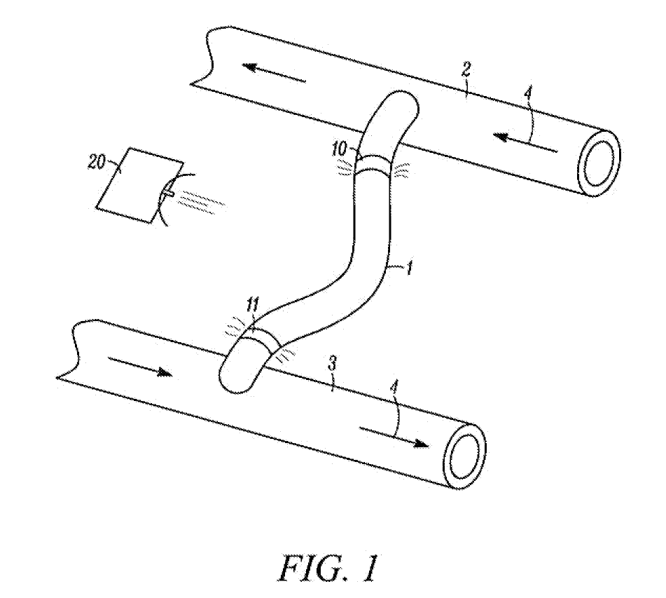

Now, referring to the drawings, FIG. 1 is a perspective view of an embodiment of a UV glowing graft 1. The graft 1 is positioned between an artery 2 and a vein 3. Arrows 4 within the artery 2 and vein 3 indicate the direction of blood flow within that vessel. In this embodiment an artery needle insertion site 10 and a vein insertion site 151 are indicated as glowing bands. In this embodiment only the bands are made or coated with a UV fluorescing material and thus can be the same or different color. The bands in this embodiment are depicted as glowing but would need to have a UV source of light 20 shined on the patient for the bands to continue to glow. The bands could also be reinforced as needed since it is intended that there will be multiple needle sticks into this region of the graft.

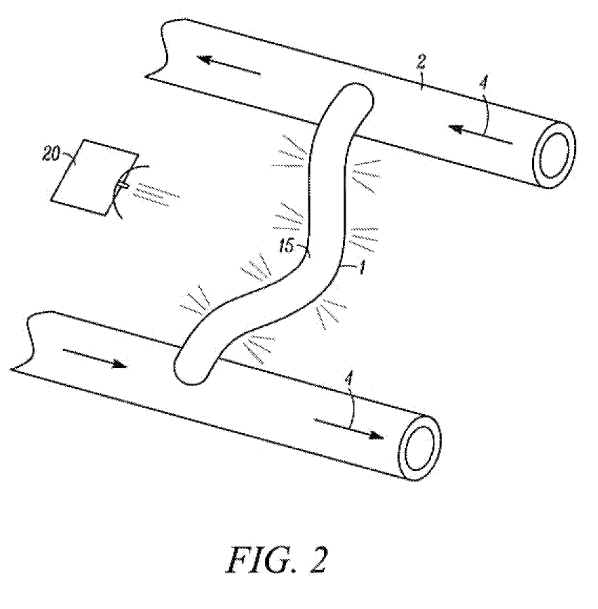

FIG. 2 is a perspective view of another graft 1 of the present invention. In this perspective graft 1 is made entirely of UV glowing polymer 15 such that upon exposure to the UV light 20, the entire length of the graft 1 will glow. In this embodiment it would likely be that a single color would be impregnated into the polymer used for the graft. Other features know for other grafts could be included as well; however, the main feature of UV glowing polymer would remain the same. One skilled in the art in view of this disclosure could easily pick colors of polymers that fluoresce as well as means of combining multiple colors and colors at particular locations in the graft. The disclosure in the specification and the claims which follow the specification are to be read broadly and not intended to be limited by any specific example or embodiment herein.

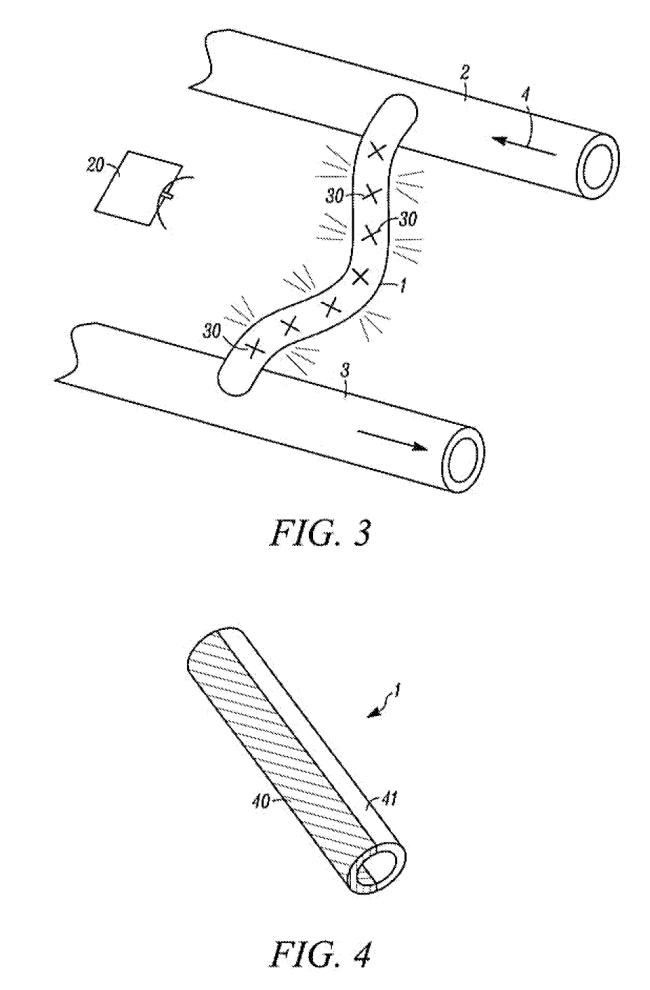

FIG. 3 shows an embodiment of the invention where graft 1 has multiple stick sites indicated by X 30. Each X 30 is made of a UV fluorescent material to indicate where to inject the needle but by giving them a distinctive shape they become easy to find. Clearly, the shape could be other than the X30 indicated and could be other shapes such as logos, other letters, bulls eyes, numbers or the like. One using this embodiment could rotate through each of the stick sites and then start again so as not to over stress any particular site by multiple injections.

FIG. 4 shows a different embodiment. In this embodiment the graft 1 is made of 2 different UV fluorescent polymers. The front wall 41 is made of a first colored polymer and the back wall 40 is made of a second different color. This format for producing the present invention has a distinct advantage so that when cannulating it makes it easier to prevent the user from puncturing the back wall or to see if it happens to prevent problems associated with passing a needle all the way through the graft.

As can be seen from the embodiments above as well as in the figures, various embodiments can clearly be chosen varying the color location and the like of the UV polymer in the graft of the present invention. Variations other than those embodiments described are within the skill of the art in view of the disclosure, and those such embodiments are within the scope of the claims which follow.

What is claimed is:

1. (canceled)

2. (canceled)

3. (canceled)

4. (canceled)

5. (canceled)

6. (canceled)

7. (canceled)

8. A method for a medical professional to access an arteriovenous graft implanted in a patient for insertion of a needle comprising:
a) selecting an arteriovenous polymeric graft at least a portion of which comprises a UV fluorescing material wherein the UV fluorescing material is positioned such that, upon exposure to a UV light source, the graft or a portion of the graft fluoresces sufficiently to improve the visibility of the location of the graft for insertion of a needle;
b) positioning the graft in the patient;
c) applying a UV light source to the area where the graft is positioned; and
d) identifying an access point in the graft for insertion of the needle by observing the fluorescing graft.

9. A method for a medical professional to access an arteriovenous graft implanted in a patient for insertion of a needle comprising:
a) selecting a patient having an arteriovenous polymeric graft at least a portion of which comprises a UV fluorescing material, wherein the UV fluorescing material is positioned such that, upon exposure to UV light source, the graft or portion of the graft fluoresces sufficiently to improve visibility of the location of the graft for insertion of a needle;
b) applying a UV light source to the area where the graft is positioned;
c) identifying a place to insert a needle by observing the fluorescing graft.
